(19) United States
(12) Patent Application Publication
(10) Pub. No.: US 2011/0082124 A1
(43) Pub. Date: Apr. 7, 2011

(54) DETERGENT COMPOSITIONS FOR TREATMENT OF LIPOMAS AND OTHER CONDITIONS

(75) Inventors: Craig G. Burkhart, Toledo, OH (US); Craig N. Burkhart, Chapel Hill, NC (US)

(73) Assignee: Kythera Biopharmaceuticals, Inc.

(21) Appl. No.: 12/962,505

(22) Filed: Dec. 7, 2010

Related U.S. Application Data
(63) Continuation of application No. 11/710,601, filed on Feb. 23, 2007.
(60) Provisional application No. 60/776,095, filed on Feb. 23, 2006.

(51) Int. Cl. A61K 31/575; A61P 35/00; A61P 17/02

(52) U.S. Cl. 514/171; 514/182

(57) ABSTRACT

A method of treating a lipoma involves contacting the lipoma with a detergent to dissolve the lipoma. For example, the detergent may be a cholate, deoxycholate or similar detergent. A method of treating cellulite and extra fat deposits involves contacting them with a detergent to help the dissolving of the fat tissues. A method of improving the art of lipoma and extra fat removal involves using certain detergents and controlling the pH, ionic forces and/or other factors to improve detergent action.

### CROSS-REFERENCE TO RELATED APPLICATION

This application claims the benefit of U.S. Provisional Application No. 60/776,095, filed Feb. 23, 2006, the disclosure of which is incorporated herein by reference.

### BACKGROUND OF THE INVENTION

This invention relates in general to the treatment of lipomas and other conditions of the body.

A lipoma is a growth of fat cells in a thin, fibrous capsule usually found just below the skin. Lipomas are found most often on the torso, neck, upper thighs, upper arms, and armpits, but can occur almost anywhere in the body. One or more lipomas may be present at the same time. Lipomas are the most common noncancerous soft tissue growth.

The cause of lipomas is not completely understood, but the tendency to develop them is inherited. A minor injury may trigger the growth. Being overweight does not cause lipomas.

Various methods have been used to treat lipomas, such as excision, liposuction, protein injection, and sonar waves. There is still a need for improved products and methods for treating lipomas and other conditions.

### SUMMARY OF THE INVENTION

This invention relates to a method of treating a lipoma involves contacting the lipoma with a detergent to dissolve the lipoma. In one embodiment the detergent includes a cholate, deoxycholate or similar detergent.

In another embodiment, the invention relates to a method of treating cellulite and extra fat deposits with a detergent to help the dissolving of the fat tissues.

In another embodiment, the invention relates to a method of improving the art of lipoma and extra fat removal by using certain detergents and controlling the pH, ionic forces, and other factors to improve detergent action.

Various advantages of this invention will become apparent to those skilled in the art from the following detailed description of the preferred embodiments.

### DETAILED DESCRIPTION OF THE PREFERRED EMBODIMENTS

The present invention relates to the use of a product including a detergent in the treatment of lipomas and other conditions of the body. Any suitable detergent or a combination of different detergents can be used. In a preferred embodiment, the detergent used in the product is described in U.S. Pat. No. 6,417,179 to Burkhart et al., issued Jul. 9, 2002, which is incorporated by reference herein.

The detergent is selected from anionic detergents, cationic detergents, zwitterionic detergents, ampholytic detergents, amphoteric detergents, nonionic detergents, or particularly nonionic detergents having a steroid skeleton. Mixtures of such detergents can also be used. The detergent can be synthetic, natural, or semi-synthetic.

Suitable anionic detergents may include, but are not limited to, the following: long-chain (fatty) alcohol sulphates; alkali metal soaps, RCOOX, where X is sodium, potassium or ammonium, and R has a chain length between C.sub.10 and C.sub.20; alkyl aryl sulphonates; sulphonated olefins; sulphated monoglycerides; sulphated ethers; sulphated polyoxyethylated alcohols; sulphated oils; sulphosuccinates; sulphonated methyl esters; alkane sulphonates; phosphate esters; alkyl isethionates; acyl sarcosides; alkyl taurides; and fluorosurfactants. Some specific examples include sodium deoxycholate, sodium dodecyl sulphate, potassium laurate, hexadecylsulphonic acid, and sodium dioctylsulphosuccinate. In general, anionic detergents are preferred for use in the product.

Suitable cationic detergents may include, but are not limited to, the following: hexadecyl(cetyl)trimethylammonium, dodecylpyridinium chloride, dodecylamine hydrochloride, cetyl-trimethyl-ammonium-bromide (e.g., Cetrimide B.P.), and benzalkonium chloride.

Suitable zwitterionic detergents may include, but are not limited to, the following: Zwittergent 3-08(n-octyl-N,N-dimethyl-3-ammonio-1-propanesulfonate), Zwittergent 3-10(n-decyl-N,N-dimethyl-3-ammonio-1-propanesulfonate), Zwittergent 3-12(n-dodecyl-N,N-dimethyl-3-ammonio-1-propanesulfonate) (Calbiochem, LaJolla, Calif.), and betaine and betaine-like detergents wherein the molecule contains both basic and acidic groups which form an inner salt giving the molecule both cationic and anionic hydrophilic groups (e.g., as disclosed in U.S. Pat. Nos. 2,082,275, 2,702,279 and 2,255,082).

Ampholytic and amphoteric detergents can be either cationic or anionic depending on the pH of the solution. An example of an ampholytic detergent that may be suitable in the ear wax solution is N-dodecyl-N,N-dimethyl betaine. An example of an amphoteric detergent that may be suitable is alkyl dimethylamine betaine (e.g., Empigen B B from Albright & Wilson, Richmond, Va.). Other nonlimiting examples of amphoteric and ampholytic detergents that may be suitable are dodecylbeta-alanine, N-alkyltaurines, N-higher alkylaspartic acids, and the detergents sold under the trade name “Miranol”, and described in U.S. Pat. No. 2,528,378.

Preferably, the detergent is selected from the category of detergents having a steroid skeleton. Anionic detergents having a steroid skeleton may include, but are not limited to, the following: sodium deoxycholate, sodium cholate, sodium taurocholate, and sodium taurodeoxycholate. Nonionic detergents having a steroid skeleton may include, but are not limited to, the following: N,N-Bis(3-D-gluconamidopropyl)cholamide (e.g., BIGCHAP, Dojindo Molecular Technologies, Gaithersburg, Md.), N,N-Bis(3-D-gluconamidopropyl)deoxycholamide (e.g., DeoxyBIGCHAP), and digitonin. Zwitterionic detergents having a steroid skeleton may include, but are not limited to, the following:, 3[(3-Cholamidopropyl) dimethylammonio]propanesulfonic acid (e.g., CHAPS). Other categories of detergents having a steroid skeleton may also be suitable.

More preferably, the detergent having a steroid skeleton is a natural, semi-synthetic, or synthetic bile salt. Naturally occurring bile salts are biological detergents synthesized in the liver. The commonly occurring bile acids include cholic acid, deoxycholic acid, lithocholic acid, chenodeoxycholic acid, hyodeoxycholic acid, and hyocholic acid. The bile acid can be a primary or secondary bile acid. The bile salts include alkali metal salts of such acids, such as sodium deoxycholate and sodium cholate.

Other examples of naturally occurring detergents that may be used in the ear wax solution include phosphatides which are surface-active agents, such as lecithin and dialkylglycerylphosphorylcholine.

In another preferred embodiment, the product includes a plurality of detergents. For example, the product may contain a mixture of sodium deoxycholate and sodium dodecyl sulphate.

The detergent can be used in any suitable amount in the product. Preferably, the product includes from about 0.5% to about 10% by weight of the detergent, more preferably from about 1% to about 5%.

In some embodiments, the product also includes a solvent for the detergent. Any suitable solvent can be used. In some embodiments, the solvent is water, a hydrophilic solvent, or a mixture thereof. Examples of hydrophilic solvents include alkylalcohols such as isopropanol, methanol, ethanol, n-propanol, n-butanol, secondary butanol, tertbutanol and isobutanol, alkylene glycols such as propylene glycol and polyethylene glycol, ether alcohols such as methyl cellosolve, ethyl cellosolve, propyl cellosolve, butyl cellosolve, methyl carbitol and ethyl carbitol, ether esters such as methyl cellosolve acetate and ethyl cellosolve acetate, dioxane, dimethylformamide, diacetone alcohol, methyl ethyl ketone, acetone, tetrahydro-furfuryl alcohol, and mixtures thereof.

In some embodiments, the product also includes an alkaline material effective to make the product. Any suitable alkaline material can be used. Some examples of alkaline materials include the sodium, potassium, calcium, magnesium and aluminum salts of phosphoric acid, carbonic acid, citric acid, and certain aluminum/magnesium compounds. Other examples include antacid materials such as aluminum hydroxides, calcium hydroxides, magnesium hydroxides and magnesium oxide. A preferred alkaline material for use in the product is disodium phosphate. Any suitable amount of alkaline material can be used in the product. Generally, the amount of alkaline material in the product is between about 0.1% and about 5% by weight of the product.

In some embodiments, the product also includes an ionic additive effective to increase the ionic strength of the product. Any suitable ionic additive can be used in the product. For example, the ionic additive may be an alkali metal salt, such as an alkali metal salt of a halogen. Some examples of suitable ionic additives include sodium chloride, potassium chloride, sodium bromide, potassium bromide, sodium iodide, potassium iodide and the like. Any suitable amount of ionic additive can be used in the product. Generally, the amount of ionic additive in the product is between about 0.1% and about 5% by weight of the product.

In some embodiments, the product also includes a polymer delivery system. The polymer can be water soluble, or non-water soluble, and can come in various lengths to accommodate a particular product and its use. Some particular polymers are disclosed in U.S. Pat. No. 6,417,179. The polymer can be used in any suitable amount. Generally, the amount of polymer in the product is between about 0.5% and about 20% by weight of the product, and typically between about 0.5% and about 10%.

The product can be formulated to have any suitable pH. In some embodiments, the product has a pH within a range of from about 5 to about 11.5.

The product can be applicable to numerous clinical situations. Uses for this product may include dissolving lipomas, fat, mesotherapy, separating tissue, tumor reduction, cancer reduction, cancer treatment, and any other clinical situation where one might want to use loosen, remove, assist the body consumption or resolution of wax, lipids, proteins, or carbohydrates from a part or region of the body. For example, in the treatment of lipomas, the product can be injected injected subcutaneously in contact with the lipomas to dissolve the lipomas. Any other suitable method of application to the lipomas can also be used.

The product may be useful in the treatment of granulomas, scars, tumors, acne cysts, sebaceous cysts, sebaceous hyperplasia, diseases of the sebum, acne related dermatoses, diseases of the subcutaneous fat, tumors, tattoo removal, infections and biofilms, cellulite, fatty deposits, fat tissue, and related conditions. It could also be used to even out skin contour defects (such as breast asymmetry, or lip assymmetry, for example, after over correction with a skin filler, or a hyperresponse of the body to an injection). Such a procedure could be done in living tissue, tissue removed from the body, or for assistance in pathological or detective assessments. It could be done on tissue, serum, or any other body partition. In addition to human care, the product is also applicable to animal care.

In accordance with the provisions of the patent statutes, the principle and mode of operation of this invention have been described in its preferred embodiments. However, it must be understood that this invention may be practiced otherwise than as specifically described without departing from its spirit or scope.

1-4. (canceled)

5. A method for treating or reducing a tissue disorder which method comprises contacting the tissue disorder with a composition comprising a detergent wherein said tissue disorder is selected from the group consisting of granulomas, scars, tumors, cancer, acne cysts, acne-related dermatoses, sebaceous cysts, and sebaceous hyperplasia.

6. The method according to claim 5, wherein said detergent is a natural, semi-synthetic, or synthetic bile acid.

7. The method according to claim 6, wherein said bile acid is deoxycholic acid or an alkali salt thereof.

8. The method according to claims 7, wherein said deoxycholic alkali salt is the sodium deoxycholate.

9. The method according to claim 5, wherein said detergent comprises a plurality of different detergents.

10. The method according to claim 9, wherein said plurality of detergents comprises sodium deoxycholate and sodium dodecyl sulphate.

11. The method according to claim 5, wherein said detergent in said composition is from about 0.5 to about 10% by weight.

12. The method according to claim 11, wherein said detergent in said composition is from about 1 to about 5% by weight.

13. The method according to claim 5, wherein contacting is made in a living tissue or a tissue removed from a body.
